(19) United States
(12) Patent Application Publication
(10) Pub. No.: US 2008/0132900 A1
(43) Pub. Date: Jun. 5, 2008

(54) Drill alignment assembly for a bone plate using tissue protection sleeves that are fixed in the bone plate

(75) Inventors: Ole Prien, Kiel (DE); Axel Bernhard Cremer, Fahrenkrog (DE)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Stryker Trauma GmbH, Schonkirchen (DE)

(21) Appl. No.: 11/985,048

(22) Filed: Nov. 13, 2007

Related U.S. Application Data
(60) Provisional application No. 60/858,794, filed on Nov. 13, 2006.

(51) Int. Cl. A61B 17/58

(52) U.S. Cl. 606/96

(57) ABSTRACT

A tissue protection sleeve for use with a drill alignment assembly for a bone plate includes a tubular body having a first end and a second end. A hole extending from the first end to the second end is formed in the tubular body. The hole has a first center line. An expanded section is formed at the second end, the expanded section has a second center line. A first nose is formed at the first end, the first nose is adapted to fit in a screw hole in the bone plate such that the sleeve is rigidly fixed to the bone plate. The drill alignment assembly further includes an aiming arm adapted to mount to a bone plate and a drill guide insertable in the tissue protection sleeve.

### CROSS-REFERENCE TO RELATED APPLICATION

This application claims the benefit of the filing date of U.S. Provisional Patent Application No. 60/858,794 filed Nov. 13, 2006, the disclosure of which is hereby incorporated herein by reference.

### BACKGROUND OF THE INVENTION

1. Field of the Invention

The present invention relates to tissue protection sleeves for use in a drill alignment assembly for bone plate. Particularly, the present invention relates to tissue protection sleeves that protect the soft tissue during the drilling of bone and shield the bone plate and the tissue from lateral forces, and allow accurate alignment of a tool with a hole in the bone plate.

2. Description of the Prior Art

A drill alignment assembly for a bone plate provides a platform for accurately locating and drilling screw holes in bone. The conventional tissue protection sleeves used in the drill alignment assemblies do not achieve the desired accuracy in locating the holes on the bone. Additionally, the conventional tissue protection sleeves can move on the surface of the bone plate and thereby allow lateral forces from the tools to act on the bone plate and tissue.

A conventional drill alignment assembly includes an aiming arm positioned over the bone plate; the aiming arm having several alignment bores for receiving and guiding a drill guide to the center of the screw holes in the bone plate. The conventional drill alignment assembly does not really accommodate for mis-alignments which can occur between the alignment bores in the aiming arm and the screw holes in the bone plate, e.g., in areas where the bone plate has been shaped or bent to conform to the contours of the bone surface. In such circumstances, adjustment of one or both structures is needed to provide correct drill placement, further extending the procedure time of the operation.

Thus, there is a need for an improved tissue protection sleeve that would allow drilling of the holes in the bone with enhanced accuracy and without forces from the tool being transmitted to the bone plate and the tissue.

### SUMMARY OF THE INVENTION

The tissue protection sleeve of the present invention overcomes the shortcomings of the prior art. The tissue protection sleeve for use with a drill alignment assembly for a bone plate includes a tubular body having a first end and a second end. A hole extending from the first end to the second end is formed in the tubular body. The hole has a first center line. An expanded section is formed at the second end, the expanded section has a second center line. A first nose is formed at the first end, the first nose is adapted to fit in a screw hole in the bone plate such that the sleeve is rigidly fixed to the bone plate.

A drill alignment assembly of the present invention includes an aiming arm adapted to mount to a bone plate. The aiming arm has alignment bores, and the bores having a first longitudinal center line. A tissue protection sleeve having a hole is provided. The hole has a second longitudinal center line. The tissue protection sleeve is adapted for insertion in any one of the alignment bores. Also included is a drill guide insertable in the tissue protection sleeve. The drill guide has an opening adapted to receive and guide a tool. The opening has a third longitudinal center line. A first nose is formed at a first end of the tissue protection sleeve. The first nose is adapted to fit in a screw hole in the bone plate such that the sleeve is rigidly fixed to the bone plate. A second nose may also be formed at the first end of the tissue protection sleeve.

### BRIEF DESCRIPTION OF THE DRAWINGS

FIG. 1 shows an exemplary bone plate drill alignment assembly.

FIG. 1A is a top view of an embodiment of a tissue protection sleeve.

FIG. 1B is an end view of the tissue protection sleeve of FIG. 1A.

FIG. 1C is a side view of the tissue protection sleeve of FIG. 1A.

FIG. 1D is an end view of the tissue protection sleeve of FIG. 1C.

FIG. 1E is a sectional view of the tissue protection sleeve of FIG. 1A.

FIG. 1F is a top view of another embodiment of a tissue protection sleeve.

FIG. 1G is an end view of the tissue protection sleeve of FIG. 1F.

FIG. 2A is a top view of an embodiment of a tissue protection sleeve.

FIG. 2B is a cross-sectional view of the tissue protection sleeve of FIG. 2A.

FIG. 2C is a side view of another embodiment of a tissue protection sleeve. FIG. 2D is an end view of a tissue protection sleeve of FIG. 2C.

FIG. 3A illustrates details of an embodiment of the drill alignment assembly shown in FIG. 1.

FIG. 3B illustrates a cross-sectional view of a portion the drill alignment assembly of FIG. 1.

FIG. 4A is an isometric view of an embodiment of the tissue protection sleeve of present invention.

FIG. 4B illustrates location of various center lines when the tissue protection sleeve is in two different positions.

### DETAILED DESCRIPTION

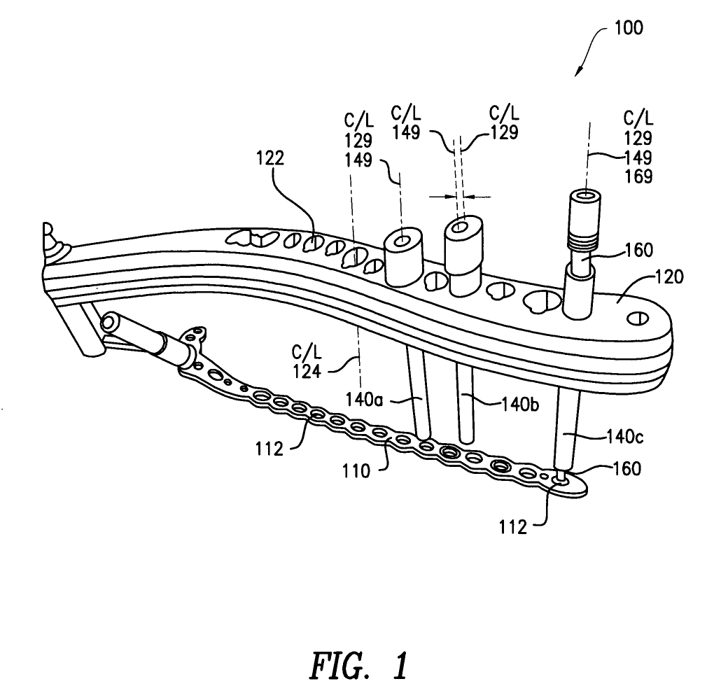

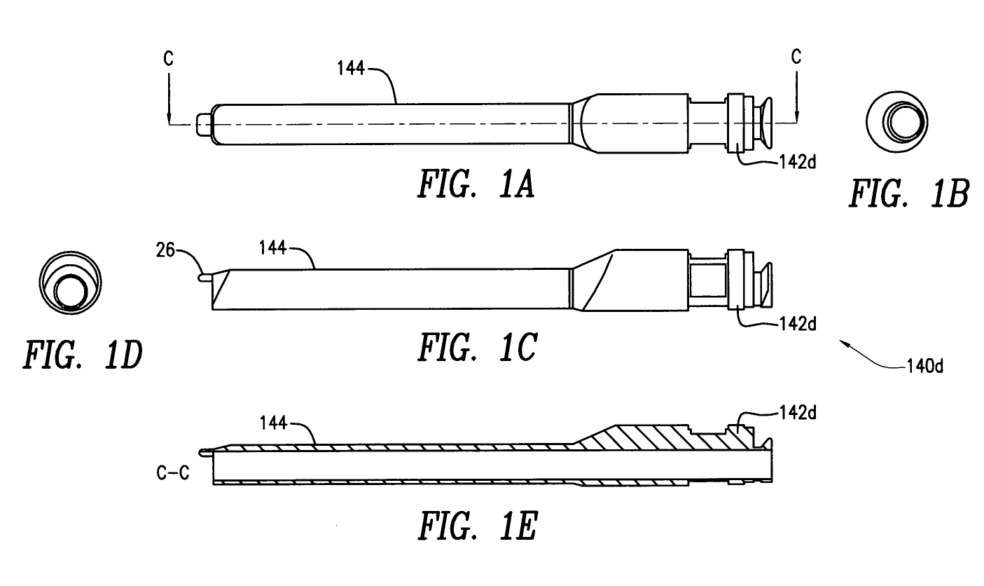

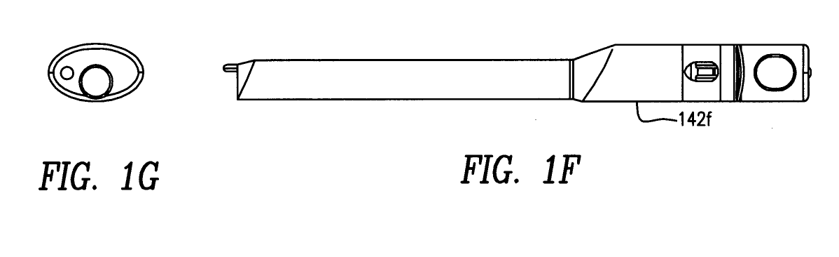

FIG. 1 illustrates an exemplary bone reconstruction plate drill alignment assembly in accordance with the present invention. As shown, the assembly 100 includes a bone reconstruction plate 110 (“bone plate” hereafter), a radiolucent aiming arm 120, a tissue protection sleeve 140 (three exemplary embodiments 140a, 140b, and 140c shown), and a drill guide 160. The bone plate 110 is adapted for attachment to a bone, and includes one or more holes 112 for receiving screws, or other attachment means operable to secure the bone plate 110 to a bone. The opening of the screw holes 112 may be generally circular in shape, or they may be non-circular, e.g., elongated/elliptical in shape. The bone plate 110 may be formed from a variety of materials, such as surgical stainless steel, Ti, or other suitable metals or materials.

The aiming arm 120 includes one or more alignment bores 122 for guiding the drilling apparatus (or any other appropriate tool) into the desired target location within a screw hole 112. Each of the alignment bores 122 defines a center line 129 which is substantially aligned with (i.e., on boresight with) the center of the screw holes 112. The aiming arm 120 may be constructed from an X-ray transparent type of material, for example, injection molded thermoplastic containing, e.g., 70% Poly Ether Ether Ketone (PEEK) and 30% carbon fibers.

The assembly 100 further includes a tissue protection sleeve 140 operable for insertion into an alignment bore 122, and a drill guide 160 operable for insertion into the tissue protection sleeve 140. The tissue protection sleeve and drill guide 160 operate to position the drilling apparatus to the desired target drilling location within a screw hole 112.

Three exemplary tissue protection sleeves 140a, 140b, and 140c are shown in FIG. 1. In the first example 140a, the center line 149 of the tissue protection sleeve is aligned with the center line 129 of the alignment bore 122. In the second example, the tissue protection sleeve center line 149 is offset from the center line 129 of the alignment bore 122. Aligning the tissue protection sleeve center line 149 offset from the alignment bore center line 129 may be advantageous to correct mis-alignment between the bone plate 110 and the aiming arm 120, whereby the offset alignment places the drilling target on boresight within the desired screw hole 112. In another embodiment, when the alignment bore center line 129 is on-boresight within the screw hole, aligning the tissue protection sleeve 140b center line 149 offset from the alignment bore center line 129 may also be advantageous in order to provide a more secure attachment to bone plate attachment point. The third example of the tissue protection sleeve 140c illustrates mutual alignment of the alignment bore, tissue protection sleeve and drilling bore center lines 129, 149 and 169 respectively. The specific construction of the tissue protection sleeve 140 is further described below.

FIGS. 1A, 1B, 1C, 1D and 1E show a fourth exemplary tissue protection sleeve 140d. The tissue protection sleeve 140d has a noose 26 formed at its end that is adjacent to the bone plate 110 when the tissue protection sleeve 140d is in use. Tissue protection sleeve 140d has an outer barrel 144 and a securing top 142d. Nose 26 may be included in any embodiment of the tissue protection sleeve 140 including tissue protection sleeve 140a, 140b and 140c. The securing top 142d may incorporate various features described in the context of other embodiments of the tissue protection sleeve 140. FIGS. 1F and 1G show yet another embodiment of tissue protection sleeve 140. The embodiment of FIGS. 1F and 1G has construction similar to the embodiment of FIG. 1A except that the securing top 142f of the embodiment of FIGS. 1F and 1G has an oval cross-section.

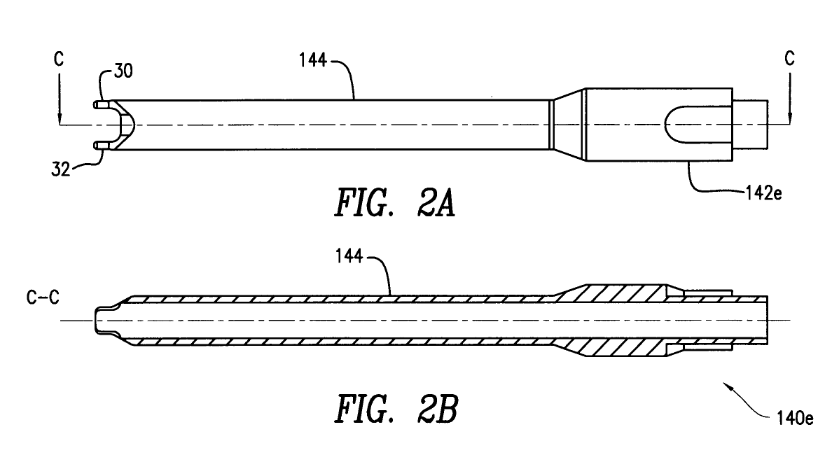

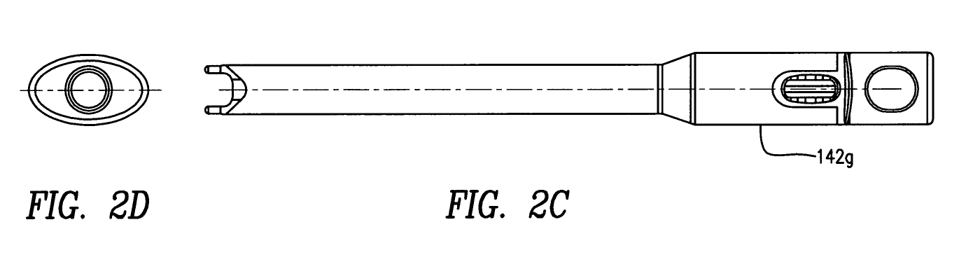

FIGS. 2A and 2B show a fifth exemplary tissue protection sleeve 140e. The tissue protection sleeve 140e has two noses 30 and 32 formed at its end that is located adjacent to the bone plate 110 when the tissue protection sleeve 140e is in use. Noses 30 and 32 may be included in any embodiment of the tissue protection sleeve 140 including tissue protection sleeve 140a, 140b and 140c. Tissue protection sleeve 140e has an outer barrel 144 and a securing top 142e. The securing top 142e may incorporate various features described in the context of other embodiments of the tissue protection sleeve 140. FIG. 2C shows yet another embodiment of tissue protection sleeve 140. The embodiment of FIG. 2C has construction similar to the embodiment of FIG. 2A except that the securing top 142g of the embodiment of FIG. 2c has an oval cross-section.

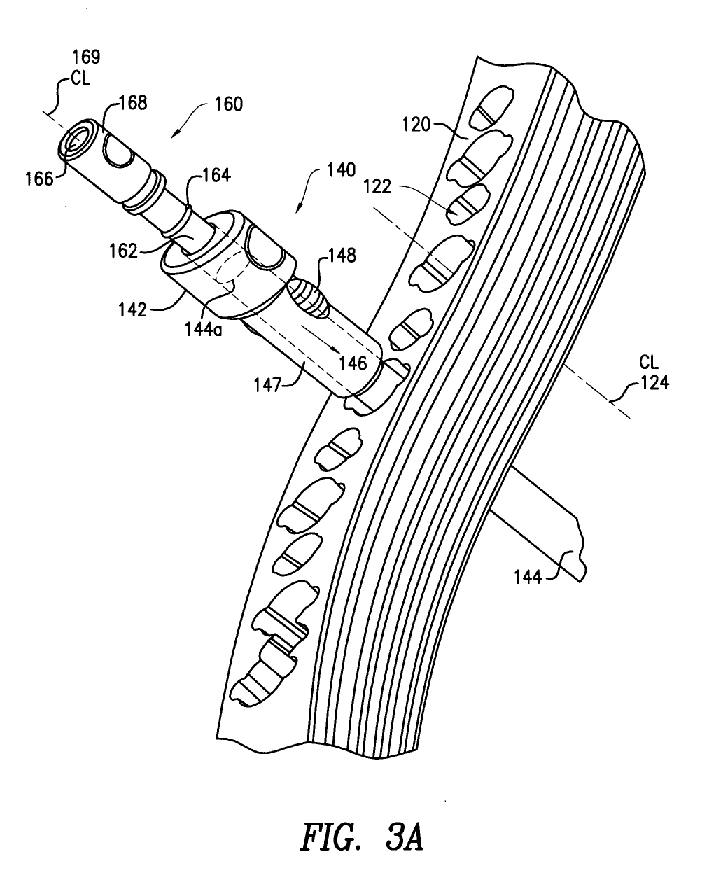

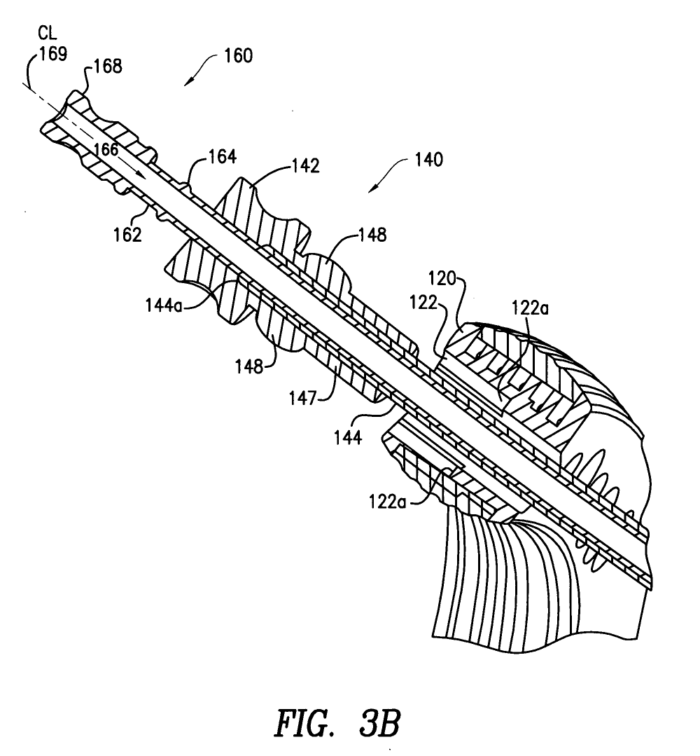

FIG. 3A illustrates details of an embodiment of the drill alignment assembly shown in FIG. 1, with previously identified features retaining their reference numerals. The tissue protection sleeve 140 is shown inserted into an alignment bore 122, and a drill guide 160 inserted into the tissue protection sleeve 140. The tissue protection sleeve 140 includes a securing top 142 which is formed on an outer barrel 144, whereby a tissue protection sleeve bore 146 extends through each of the securing top 142 and the outer barrel 144. The tissue protection sleeve 140 further includes an engagement sleeve 147, which in a particular embodiment has an outer diameter which is substantially that of the hole diameter of the alignment bore 122 into which the tissue protection sleeve 140 is to be press-fit. In such an embodiment, the outer diameter of the engagement sleeve 147 defines substantially the alignment bore center line 129. The tissue protection sleeve 140 further includes one or more locking members 148 operable to provide a press-compression fit within the alignment bore 122.

The drill guide 160 may be inserted into the tissue protection sleeve bore 146, and includes a drill guide barrel 162, and an engagement member 164 disposed on the drill guide barrel's outer surface. A drill guide bore 166 for receiving a drill bit extends through the drill guide barrel 162 along a center line 169. In particular, the engagement member 164 is operable to provide a press compression fit within the securing top 142 of the tissue protection sleeve to thereby secure the drill guide 160 to the tissue protection sleeve 140. In this manner, the assembly can be moved and turned upside down without the drill guide 160 disengaging from the tissue protection sleeve 140. Drill guide grip 168 facilitates removal of the drill guide 160 from the tissue protection sleeve 140.

In a particular embodiment, the securing top 142 is formed from an elastic material, such as rubber or similarly resilient materials. It will be understood that such materials will be able to provide a press compression fit operable to hold the drill guide 160 into the tissue protection sleeve 140 during movement of the assembly, while enabling the drill guide's removal from the tissue protection sleeve 160 with only a small increase in pulling force.

The tissue protection sleeve 140 may further include an engagement member stop 144a which is operable to inhibit further travel or insertion of the drill guide 160 into the tissue protection sleeve 140. Further travel of the drill guide 160 into the tissue protection sleeve 140 could result in the drill guide barrel 162 forcefully impacting the bone plate 110, resulting in mis-alignment of the drilling process, or damage to the drill guide barrel 162 and/or bone plate 110. In a specific embodiment, the engagement member 164 is formed as a raised annular ring extending around the outer surface of the drill guide barrel 162, and the engagement member stop 144a provides a diameter which is greater than the outer diameter of the drill guide barrel 162 to permit passage of the outer surface therethrough, but which is smaller than the clearance needed for the raised annular ring 164. This arrangement will prevent further travel of the drill guide 160 into the tissue protection sleeve 140.

FIG. 3B illustrates a cross-sectional view of a portion the drill alignment assembly 100 with previously identified features retaining their reference numerals. As illustrated, the tissue protection sleeve 140 is formed so as to make a press compression fit into a bore 122 of the aiming arm 120. One or more resilient locking members 148 are formed onto the engagement sleeve 147 of the tissue protection sleeve 140, the locking members 148 formed generally complementary to a cut-out pattern in the bores 122 of the aiming arm 120, the engagement sleeve 147 and the locking members 148 composed of a resilient elastic material. Upon insertion of the tissue protection sleeve 140 into an alignment bore 122, the engagement sleeve 147 and the resilient locking members 148 become depressed to form a press compression fit within the bore 122, thereby retaining the tissue protection sleeve 140 therein. A locking member stop 122a disposed within the alignment bore 122 (shown as a counter-sunk diameter bore included within alignment block bore 122) is used to stop further travel of the engagement sleeve 147 and the locking members 148 into the alignment bore 122. Removal of the tissue protection sleeve 140 from the alignment block 122 is effectuated by supplying sufficient force (e.g., upward force) to overcome the compression fit. Each of the tissue protection sleeve 140 and the drill guide 160 may include grips formed at a convenient location of their respective structures.

As noted above and illustrated in FIG. 1, the drill guide bore 166 and the alignment bore 122 may be centered with respect to each other (i.e., center lies 129 and 169 are mutually aligned), or alternatively, arranged in an offset manner. Alignment of the drill guide and alignment bore center lines 129 and 169 is advantageous when the alignment bore is substantially on-boresight with the center of the screw hole 112. However, some mis-alignment between the screw hole 112 and the aiming arm 120 can occur, in which case the center line 124 may not be sufficiently on boresight of the screw hole 112. In this case, an adjustment of the drill guide 160 to provide a matching offset is needed in order to place a drill guide center line 169 on boresight with the mis-aligned screw hole.

In another embodiment, the drill guide center line 169 may be sufficiently on-boresight with the screw hole 112, however, offset placement of the drilled hole from the screw hole is desired in order to provide a more advantageous screw position. Such an offset placement may be needed in order to provide an attachment point through a bent portion of the bone plate 110. As noted above, the screw holes 112 can be made elongated or elliptical in their opening to accommodate different screw positions.

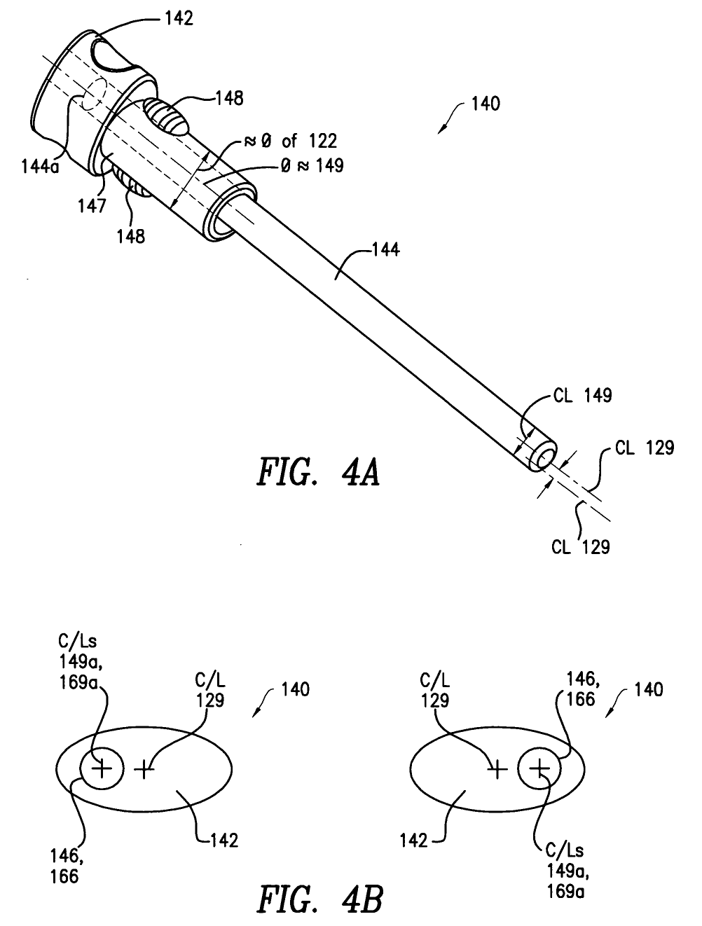

FIG. 4A illustrates a tissue protection sleeve 140 configured in accordance with one embodiment of the present invention. As shown, the tissue protection sleeve 140 is operable to provide a center line 149 which is offset from the center line 129 of the alignment bore. The tissue protection sleeve 140 includes an engagement sleeve 147 having an outer diameter which is substantially that of the hole diameter of the alignment bore 122 into which the tissue protection sleeve 140 is to be press-fit. Accordingly, the outer diameter of the engagement sleeve 147 defines substantially the center line 129, the center line 129 of the alignment bore 122 being substantially aligned with the center line of the screw hole 112.

The tissue protection sleeve 140 further includes a bore 146 which extends through the securing member 142 and the tissue protection sleeve barrel 144, defining a center line 149. The tissue protection sleeve bore 146 is operable to receive the drill guide 160, the construction of the drill guide 160 being substantially as described above. When inserted into the tissue protection sleeve 140, the drill guide 160 will have a center line substantially that of the center line 149. Accordingly, the center line 169 of the drill guide 160 will be offset from the center line of the screw hole 112. In this manner, securely aligning the drill guide 160 to drill holes for an offset screw can be accomplished.

In an extension of the foregoing, the tissue protection sleeve 140 can be rotated 180 degrees to provide a center line 149 on the opposite side of the alignment bore center line 129. FIG. 4B illustrates this arrangement in which a tissue protection sleeve 140 is shown in two positions, one rotated 180 degrees relative to the other. The left most illustration shows the tissue protection sleeve 140 having a center line 149a disposed to the left side of the alignment bore center line 129, and the right most illustration shows the tissue protection sleeve 140 having a center line 149b disposed to the right side of the alignment bore 129. The tissue protection sleeve bores 146 are operable to receive a drill guide 160, the drill guides 160 having respective drill guide bores 166 defining center lines 169a and 169b which are substantially that of the tissue protection sleeve bore center lines 149a and 149b. Accordingly, a screw hole which is offset in either the left or right side of the alignment bore center line 129 can be correctly aligned to. In addition, if the aiming arm 120 and the tissue protection sleeve 140 are appropriately configured to provide four different engaging positions (e.g., the aiming arm having four cut-outs within bore 122, and the tissue protection sleeve 140 having four locking members 148), the tissue protection sleeve 140 can be rotated 90 degrees to provide offset alignments in four different positions around the alignment bore center line 129. Of course, the aforementioned technique can be extended to provide a larger number of offset positions around the alignment bore center line 129.

In use, the drill alignment assembly 100 is attached to a bone plate 110 and a tissue protection sleeve 140 is inserted in an alignment bore 122 of the aiming arm 120. Next, a drill guide 160 is inserted in the tissue protection sleeve 140. If the center line of the drill guide 160 does not line up with the screw hole 112, it is brought in desired alignment using one of the techniques discussed above. Next, in case a tissue protection sleeve with a nose is used, for example, tissue protection sleeve 140d or 140e, the tissue protection sleeve is lowered in the screw hole 112. Consequently, nose 26 or noses 30 and 32 of tissue protection sleeve 140d or 140e, respectively, engage side of screw hole 112 fixing the tissue protection sleeve in the bone plate 110 and protecting the surrounding tissue from lateral forces from the tools that may be inserted in drill guide 160.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A tissue protection sleeve for use with a drill alignment assembly for a bone plate, the sleeve comprising:
a tubular body having a first end and a second end;
a hole formed in the tubular body, the hole extending from the first end to the second end, the hole having a first center line;
an expanded section formed at the second end, the expanded section having a second center line; and
a first nose formed at the first end, the first nose being adapted to fit in a screw hole in the bone plate such that the sleeve is rigidly fixed to the bone plate.

2. The tissue protection sleeve of claim 1, wherein a second nose is formed at the first end, the second nose being adapted to fit in the screw hole in the bone plate and cooperate with the first nose to rigidly fix the sleeve to the bone plate.

3. The tissue protection sleeve of claim 1, wherein the first centerline and the second centerline are at a distance from each other and are mutually parallel.

4. The tissue protection sleeve of claim 1, wherein the cross section of the second end is elliptical.

5. The tissue protection sleeve of claim 1, wherein the second end is made from a resilient material.

6. A drill alignment assembly for a bone plate, the drill alignment assembly comprising:
an aiming arm adapted to mount to the bone plate, the aiming arm having alignment bores, the bores having a first longitudinal center line;
a tissue protection sleeve adapted for insertion in any one of the alignment bores, the tissue protection sleeve having a hole, the hole having a second longitudinal center line; and
a drill guide insertable in the tissue protection sleeve, the drill guide having an opening adapted to receive and guide a tool, the opening having a third longitudinal center line, wherein
a first nose is formed at a first end of the tissue protection sleeve, the first nose being adapted to fit in a screw hole in the bone plate such that the sleeve is rigidly fixed to the bone plate.

7. The drill alignment assembly of claim 6, wherein the tissue protection sleeve includes an expanded section formed at a second end, the expanded section being adapted to have a sliding fit in the alignment bores.

8. The tissue protection sleeve of claim 6, wherein a second nose is formed at the first end, the second nose being adapted to fit in the screw hole in the bone plate and cooperate with the first nose to rigidly fix the sleeve to the bone plate.

9. The tissue protection sleeve of claim 6, wherein the first centerline, second and third center lines are aligned.

10. The tissue protection sleeve of claim 7, wherein the expanded end has a fourth center line, and the fourth centerline and the second centerline are at a distance from each other and are mutually parallel.

11. The tissue protection sleeve of claim 10, wherein the first and the second center line are aligned.

12. The tissue protection sleeve of claim 6, wherein the first centerline and the second centerline are aligned and are at a distance from the third center line and the first and second center line are parallel to the third center line.

13. The tissue protection sleeve of claim 7, wherein the cross section of the second end is elliptical.

14. The tissue protection sleeve of claim 7, wherein the second end is made from a resilient material.
